(19) United States
(12) Patent Application Publication
(10) Pub. No.: US 2005/0272032 A1
(43) Pub. Date: Dec. 8, 2005

(54) Internal control for in situ hybridization

(75) Inventor: Jay Ji, Tucson, AZ (US)

Correspondence Address: MCDONNELL BOEHNEN HULBERT & BERGHOFF LLP, 300 S. WACKER DRIVE, 32ND FLOOR, CHICAGO, IL, 60606, US

(73) Assignee: Ventana Medical Systems, Inc., a corporation of the State of Delaware

(21) Appl. No.: 11/121,521

(22) Filed: May 4, 2005

Related U.S. Application Data
(60) Provisional application No. 60/567,889, filed on May 4, 2004.

(51) Int. Cl. C12Q 1/70; C12Q 1/68; C12P 19/34

(52) U.S. Cl. 435/5; 435/6; 435/91.2

(57) ABSTRACT

The invention provides a method for monitoring the quality of in situ hybridization analysis of a nuclear DNA target in a tissue or cell sample using a mitochondrial DNA probe as an internal control. The invention also provides a reagent for in situ hybridization detection of a nuclear DNA target and a mitochondrial DNA target in a tissue or cell sample.

### BACKGROUND OF THE INVENTION

1. Field of the Invention

The invention relates to a method for monitoring the quality of in situ hybridization analysis of a nuclear DNA target in a tissue or cell sample using a mitochondrial DNA probe as an internal control. The invention also relates to a reagent for in situ hybridization detection of a nuclear DNA target and a mitochondrial DNA target in a tissue or cell sample.

2. Background of the Invention

Nucleic acid hybridization is a process in which two single-stranded nucleic acid molecules having sufficiently complementary sequences are allowed to interact under suitable reaction conditions so as to form a double-stranded nucleic acid hybrid. Hybridization techniques generally can be classified into one of three groups: (1) solution hybridization techniques, in which the hybridization reaction between the complementary, single-stranded nucleic acid molecules is carried out in solution; (2) filter or blot hybridization techniques, in which one of the single-stranded nucleic acid molecules is bound to a solid matrix prior to hybridization with a complementary single-stranded nucleic acid molecule; and (3) in situ hybridization (ISH), in which one of the single-stranded nucleic acid molecules is isolated from suitably prepared cells or histological sections, thereby allowing for the detection and localization of specific nucleic acid sequences in tissue or cellular structures (e.g., within the nucleus of a cell). ISH, therefore, has the added benefit of permitting simultaneous determination of biochemical and morphological characteristics in a cell or tissue sample being examined.

One type of ISH assay is chromogenic in situ hybridization (CISH), in which the hybridization reaction between the complementary, single-stranded nucleic acid molecules is detected using a chromogen. For example, the hybridization of a labeled nucleic acid probe to a cellular nucleic acid target can be detected using a primary antibody directed against the labeled probe, a secondary antibody-enzyme conjugate directed against the primary antibody, and a chromogen substrate that is converted into an insoluble colored precipitate upon reaction with the secondary antibody-enzyme conjugate. In contrast with other ISH assays, CISH permits the direct visualization of molecular markers under a conventional light microscope.

ISH assays have been developed for use in diagnosing cervical cancer. In one such assay, human papillomavirus (HPV) genotypes that are associated with cervical cancer are detected using a viral probe cocktail generated by nick translation and consisting of probes of approximately 200-600 basepairs in length.

ISH offers many advantages over molecular diagnostic methods, such as Southern blot hybridization or polymerase chain reaction (PCR), that require the destruction of cellular or tissue samples. In contrast with other types of nucleic acid hybridization, ISH does not require cell lysis and subsequent isolation of nucleic acid molecules from cellular or clinical samples prior to examination. Instead, the cellular or clinical sample can be deposited directly onto a slide and then hybridized with labeled probes.

As with any molecular diagnostic method, however, the verification and interpretation of ISH results depends on the use of suitable controls. For example, target and positive control probes should be prepared by similar methods and target and positive control probes should be hybridized to cellular or tissue samples and detected under the same conditions, preferably on the same slide, to allow for the monitoring of overall assay performance, including proteinase digestion for unmasking targets, nucleic acid hybridization, immunological detection, and chromogenic visualization. Slide preparation, including specimen collection and fixation, as well as the age and storage of samples, can also influence the reliability of an ISH assay.

One suitable ISH control is a probe capable of specifically binding the human Alu element. Alu sequences are short interspersed elements, typically 300 nucleotides in length. The human genome contains over 1.4 million Alu elements, which account for approximately 10% of the genome (International Human Genome Sequencing Consortium, 2001). Alu probes can be used for the evaluation of target DNA integrity during specimen collection, processing and handing of samples, and ISH assay performance. For example, improper preservation of cellular or tissue samples can result in target DNA degradation, leading to a false negative diagnostic result. Unreliable results can also be obtained through the use of defective ISH detection reagents. In general, any negative ISH result obtained for a particular target probe should be viewed as unreliable when an inadequate staining result is obtained with an Alu control probe.

The use of Alu probes as an ISH control, however, also presents several disadvantages. First, while the copy number of Alu elements in any human cell is about 1.4 million, the copy number for most diagnostic targets in ISH assays is several thousand to a million fold less. Alu elements, therefore, can be considered as an insensitive control sequence for ISH assays. Second, because Alu elements are short, interspersed sequences comprising repetitive GC-rich regions, Alu probes require different probe preparation techniques and different hybridization conditions. For example, while Alu probes can be readily prepared by chemical synthesis on an oligonucleotide synthesizer, HPV genomic probes must be prepared using enzymatic techniques (e.g., nick translation) or direct modification. Moreover, due to their different probe lengths and compositions, Alu and HPV genomic probes require particular probe hybridization conditions and washing stringencies. Finally, Alu and HPV genomic probes present additional detection difficulties in ISH assays due to the co-localization of both control and target signals to the nucleus. In practice, therefore, because ISH assays using Alu control probes must be performed on separate slides, any operational deviations in specimen preparation, handling, or hybridization between the two slides cannot be adequately controlled.

Mitochondria are small intracellular organelles responsible for energy production and cellular respiration. These organelles, which are located exclusively in the cytoplasm, possess a double-stranded circular genome of approximately 16.5 kb in length (Anderson et al., 1981, Nature 290:457-65). Individual cells possess multiple copies of the mitochondrial genome; for example, a single human muscle cell possesses between 1.6×10⁴ and 8.5×10⁴ copies (He et al., 2002, Nucleic Acids Res. 30:e68). While the mitochondrial DNA copy number among tissue and cell samples is variable, the copy number in individual cells of the same tissue or cell sample is relatively stable, varying by no more than a few fold (Veltri et al., 1990, J. Cell. Physiol. 143: 160-64 and Smith et al., 2002 Reprod. Biomed. Online 4:248-55).

Since its initial description, ISH has undergone continuous evolution in methodology and application. At present, ISH has direct applications in many areas of biomedical and clinical research including cell biology, clinical diagnosis, developmental biology, genetics, and virology. However, there remains a need in the ISH art to develop alternative ISH controls. The biological properties of mitochondria make mitocondrial DNA a suitable internal control for use in ISH assays, and more particularly, for use in HPV target detection of cervical abnormality.

### SUMMARY OF THE INVENTION

The invention provides methods for monitoring the quality of in situ hybridization analysis of a nuclear DNA target in a tissue or cell sample using a mitochondrial DNA probe as an internal control.

In one method of the invention, the quality of in situ hybridization analysis of a nuclear DNA target in a tissue or cell sample is monitored by treating the tissue or cell sample to render chromosomal and extrachromosomal DNA present therein available for hybridization to complementary sequences; contacting the tissue or cell sample with a probe composition under hybridizing conditions, wherein the probe composition comprises a nuclear DNA probe that is substantially complementary to the nuclear DNA target conjugated to a first detectable label, and a mitochondrial DNA probe that is substantially complementary to a mitochondrial DNA target conjugated to a second detectable label; washing probe that does specifically hybridize to the target from the tissue or cell sample; simultaneously assessing the degree of hybridization between the nuclear DNA probe and the nuclear DNA target and the degree of hybridization between the mitochondrial DNA probe and the mitochondrial DNA target; and comparing the degree of hybridization observed between the mitochondrial DNA probe and the mitochondrial DNA target with the expected degree of hybridization between the mitochondrial DNA probe and the mitochondrial DNA target to determine the quality of in situ hybridization analysis of the nuclear DNA target.

In another method of the invention, the quality of in situ hybridization analysis of a nuclear DNA target in a tissue or cell sample is monitored by treating the tissue or cell sample to render chromosomal and extrachromosomal DNA present therein available for hybridization to complementary sequences; contacting the tissue or cell sample with a probe composition under hybridizing conditions, wherein the probe composition comprises a nuclear DNA probe that is substantially complementary to the nuclear DNA target conjugated to a first detectable label, and a mitochondrial DNA probe that is substantially complementary to a mitochondrial DNA target conjugated to a second detectable label; washing probe that does specifically hybridize to the target from the tissue or cell sample; assessing the degree of hybridization between the nuclear DNA probe and the nuclear DNA target; assessing the degree of hybridization between the mitochondrial DNA probe and the mitochondrial DNA target; and comparing the degree of hybridization observed between the mitochondrial DNA probe and the mitochondrial DNA target with the expected degree of hybridization between the mitochondrial DNA probe and the mitochondrial DNA target to determine the quality of in situ hybridization analysis of the nuclear DNA target.

In another method of the invention, the quality of in situ hybridization analysis of a nuclear DNA target in a tissue or cell sample is monitored by treating the tissue or cell sample to render chromosomal and extrachromosomal DNA present therein available for hybridization to complementary sequences; contacting the tissue or cell sample with a probe composition under hybridizing conditions, wherein the probe composition comprises a nuclear DNA probe that is substantially complementary to the nuclear DNA target conjugated to a first detectable label, and a mitochondrial DNA probe that is substantially complementary to a mitochondrial DNA target conjugated to a second detectable label; washing probe that does specifically hybridize to the target from the tissue or cell sample; assessing the degree of hybridization between the mitochondrial DNA probe and the mitochondrial DNA target; assessing the degree of hybridization between the nuclear DNA probe and the nuclear DNA target; and comparing the degree of hybridization observed between the mitochondrial DNA probe and the mitochondrial DNA target with the expected degree of hybridization between the mitochondrial DNA probe and the mitochondrial DNA target to determine the quality of in situ hybridization analysis of the nuclear DNA target.

In another method of the invention, the quality of in situ hybridization analysis of a nuclear DNA target in a tissue or cell sample is monitored by treating the tissue or cell sample to render chromosomal and extrachromosomal DNA present therein available for hybridization to complementary sequences; contacting the tissue or cell sample with a nuclear DNA probe that is substantially complementary to the nuclear DNA target conjugated to a first detectable label; washing nuclear DNA probe that does specifically hybridize to the nuclear DNA target from the tissue or cell sample; assessing the degree of hybridization between the nuclear DNA probe and the nuclear DNA target; contacting the tissue or cell sample with a mitochondrial DNA probe that is substantially complementary to a mitochondrial DNA target conjugated to second detectable label; washing mitochondrial DNA probe that does specifically hybridize to the mitochondrial DNA target from the tissue or cell sample; assessing the degree of hybridization between the mitochondrial DNA probe and the mitochondrial DNA target; and comparing the degree of hybridization observed between the mitochondrial DNA probe and the mitochondrial DNA target with the expected degree of hybridization between the mitochondrial DNA probe and the mitochondrial DNA target to determine the quality of in situ hybridization analysis of the nuclear DNA target.

In another method of the invention, the quality of in situ hybridization analysis of a nuclear DNA target in a tissue or cell sample is monitored by treating the tissue or cell sample to render chromosomal and extrachromosomal DNA present therein available for hybridization to complementary sequences; contacting the tissue or cell sample with a mitochondrial DNA probe that is substantially complementary to a mitochondrial DNA target conjugated to a first detectable label; washing mitochondrial DNA probe that does specifically hybridize to the mitochondrial DNA target from the tissue or cell sample; assessing the degree of hybridization between the mitochondrial DNA probe and the mitochondrial DNA target; contacting the tissue or cell sample with a nuclear DNA probe that is substantially complementary to the nuclear DNA target conjugated to second detectable label; washing nuclear DNA probe that does specifically hybridize to the nuclear DNA target from the tissue or cell sample; assessing the degree of hybridization between the nuclear DNA probe and the nuclear DNA target; and comparing the degree of hybridization observed between the mitochondrial DNA probe and the mitochondrial DNA target with the expected degree of hybridization between the mitochondrial DNA probe and the mitochondrial DNA target to determine the quality of in situ hybridization analysis of the nuclear DNA target.

The invention also provides reagents for in situ hybridization detection of a nuclear DNA target and a mitochondrial DNA target in a tissue or cell sample.

One reagent of the invention is prepared by combining a nuclear DNA probe that is substantially complementary to the nuclear DNA target conjugated to a first detectable label with a mitochondrial DNA probe that is substantially complementary to the mitochondrial DNA target conjugated to a second detectable label.

Specific preferred embodiments of the present invention will become evident from the following more detailed description of certain preferred embodiments and the claims.

### BRIEF DESCRIPTION OF THE DRAWINGS

FIG. 1 shows a dinitrophenyl (DNP)-labeled nucleotide analog (DNP-dCTP) suitable for labeling probes for use in chromogenic in situ hybridization;

FIG. 2 shows a biotinylated nucleotide analog (biotin-dCTP) suitable for labeling probes for use in chromogenic in situ hybridization;

FIG. 3 shows a fluorescein-labeled nucleotide analog (fluorescein-dCTP) suitable for labeling probes for use in chromogenic in situ hybridization;

FIG. 4 shows the results of chromogenic in situ hybridization analysis for human papilloma virus (HPV) in cell lines using mitochondrial DNA as an internal control; in panels A and B, CaSki cells (panel A) or T24 cells (panel B) were prepared by CytoSpin and hybridization of HPV and mitochondrial DNA probes was detected using alkaline phosphatase (AP) and Azoic Diazo Component; in panels C and D, CaSki cells (panel C) and T24 cells (panel D) were embedded in agar and cut at 4 μm thickness and hybridization of HPV and mitochondrial DNA probes was detected using horse radish peroxidase (HRP) and 3,3′-diaminobenzidine tetrahyrdochloride (DAB); in panels E and F, hybridization of a mitochondrial DNA probe (panel E) and an HPV probe (panel F) was detected in CaSki cells in agar using AP and Azoic Diazo Component; and in panels H-J, hybridization of an HPV probe was detected using an HPV High Risk Tissue System Control Slide (Ventana Medical Systems, Inc.) in CaSki cells (panel H), HeLa cells (panel I), or T24 cells (panel J);

FIG. 5 shows the results of chromogenic in situ hybridization analysis for human papilloma virus (HPV) in clinical samples using mitochondrial DNA as an internal control; in panel A, hybridization of a mitochondrial DNA probe in kidney tissue was detected using HRP and DAB; in panel B, hybridization of a mitochondrial DNA probe in cervical tissue was detected using HRP and DAB; in panel C, hybridization of an HPV probe in a cervical lesion was detected using AP, bromochloroindolyl (BCIP), and nitroblue tetrazolium (NBT); in panel D, hybridization of a mitochondrial DNA probe in a cervical smear liquid based preparation was detected using AP and Azoic Diazo Component; and in panel E, hybridization of an HPV probe in a cervical smear liquid based preparation using AP, BCIP, and NBT.

### DETAILED DESCRIPTION OF THE INVENTION

The invention provides methods for monitoring the quality of in situ hybridization analysis of a nuclear DNA target in a tissue or cell sample using a mitochondrial DNA probe as an internal control. The invention also provides reagents for in situ hybridization detection of a nuclear DNA target and a mitochondrial DNA target in a tissue or cell sample.

By taking advantage of the fact that a cell's nucleus and mitochondria constitute distinct organelles occupying separate regions of the cytoplasm, the quality of in situ hybridization analyses of nuclear DNA targets can be monitored by using a mitochondrial DNA probe as an internal control. To monitor the quality of an ISH assay, the degree of hybridization between the extrachromosomal DNA of a tissue or cell sample and a suitable mitochondrial DNA probe (such as the mitochondrial DNA probes described in Example 2) is assessed (e.g., by visual inspection) and the degree of hybridization observed between the mitochondrial DNA probe and the mitochondrial DNA target is compared with the expected degree of hybridization between the mitochondrial DNA probe and the mitochondrial DNA target for that tissue or cell sample. When the observed degree of hybridization and the expected degree of hybridization are not significantly different, the results of the ISH analysis with respect to the nuclear DNA target can be considered to be reliable.

A mitochondrial DNA probe can be used as an internal control to monitor the quality of in situ hybridization analysis of a nuclear DNA target in a tissue or cell sample because the mitochondrial DNA copy within individual cells of the same tissue or cell sample is relatively constant. For example, Veltri et al., 1990, J. Cell. Physiol. 143: 160-64, teach that the mitochondrial DNA copy number in murine liver, kidney, heart, and brain is organ-specific. Mitochondrial DNA copy numbers for a number of other tissue and cell types, and of tissue or cell types at different developmental stages, have been published in the literature. For example, Steuerwald et al., 2000, Zygote 8: 209-15, teach that mouse and human oocytes contain an average of 1.59×10⁵ and 3.14×10⁵ mitochondrial genomes, respectively.

As used herein, the term “degree of hybridization” refers to the extent of hybridization that occurs between a labeled probe specific for a particular target and the target under suitable hybridizing conditions. One of ordinary skill in the art would understand that the degree of hybridization between a labeled probe (e.g., a mitochondrial DNA probe) and a target (e.g., mitochondrial DNA) can be assessed by determining the relative intensity or amount of the labeled probe that remains on a tissue or cell sample after the tissue or cell sample has been rinsed to remove unhybridized probes. One of ordinary skill in the art would also understand that in practicing the methods of the invention, the degree of hybridization can be assessed either qualitatively or quantitatively. For example, the degree of hybridization between a mitochondrial DNA probe and a mitochondrial DNA target may be assessed qualitatively by simple visual inspection of the tissue or cell sample following hybridization. In assessing the degree of hybridization qualititatively, one of ordinary skill in the art could rate the degree of hybridization as, for example, strong (+++), medium (++), weak (+), or none detected (−).

Alternatively, the degree of hybridization between a mitochondrial DNA probe and a mitochondrial DNA target can be assessed quantitatively by measuring the amount of the labeled mitochondrial DNA probe that hybridizes to the mitochondrial DNA target. A representative method and apparatus for quantitating protein by automated tissue staining is taught in U.S. Patent Application Publication No. 2001/0049114 A1, published Dec. 6, 2001, and entitled “Method for Quantitating a Protein by Image Analysis,” which is incorporated herein by reference in its entirety. Another slide imaging system commercially available is sold by Applied Imaging Corporation (Santa Clara, Calif.) as the ARIOL SL-50. In addition, since a number of methods have been developed for quantitating mitochondrial DNA, the mitochondrial DNA copy number for any tissue or cell type can be readily calculated in order to determine the expected degree of hybridization between a mitochondrial DNA probe and a mitochondrial DNA target. For example, Veltri et al., 1990, J. Cell. Physiol. 143: 160-64, teach a method for determining the copy number of mitochondrial DNA in cells using a radiolabelled mitochondrial DNA probe. In addition, Steuerwald et al., 2000, Zygote 8: 209-15, teach a fluorescent rapid cycle DNA amplification method for determining the number of mitochondrial genomes present in individual cells. Furthermore, Chabi et al., 2003, Clin. Chem. 49: 1309-17, teach a quantitative PCR assay for determining the copy number of mitochondrial DNA in individual cells. In the method of Chabi et al., a calibration curve is generated from serial dilutions of cloned mitochondrial DNA probes specific to four different mitochondrial genes, each of which is localized to different regions of the mitochondrial genome. The mitochondrial DNA content of various cell types could be determined using these, and other suitable methods, together with a tissue array, such as the Human Body Tour Tissue Array (City of Hope; Duarte Calif.; U.S. Pat. No. 5,002,377), which contains 28 different human tissues.

Mitochondrial DNA probes for use in the methods and reagents of the present invention may be prepared by a number of methods known to those of skill in the art. Suitable mitochondrial DNA probes may recognize any portion of the mitochondrial genome of the tissue or cell to be examined, provided that the selected probe specifically hybridizes to mitochondrial DNA. In preferred embodiments of the methods and reagents of the invention, the mitochondrial DNA probe is prepared by polymerase chain reaction using the amplimers 5′-CTC-TAG-AGC-CCA-CTG-TAA-AG-3′ (SEQ ID NO: 3) and 5′-TGA-CCG-TAG-TAT-ACC-CCC-GG-3′ (SEQ ID NO: 8). In other preferred embodiments, the mitochondrial DNA probe is prepared using the amplimers 5′-CAA-CAT-ACT-CGG-ATT-CTA-CCC-TAG-3′ (SEQ ID NO: 4) and 5′-GGG-GAA-GCG-AGG-TTG-ACC-TG-3′ (SEQ ID NO: 6); the amplimers 5′-CAA-CAT-ACT-CGG-ATT-CTA-CCC-TAG-3′ (SEQ ID NO: 4) and 5′-TGA-CCG-TAG-TAT-ACC-CCC-GG-3′ (SEQ ID NO: 8); or the amplimers 5′-CTC-TAG-AGC-CCA-CTG-TAA-AG-3′ (SEQ ID NO: 3) and 5′-GGC-AGG-AGT-AAT-CAG-AGG-TG-3′ (SEQ ID NO: 5). In still another preferred embodiment, the amplimers are 5′-AAC-ATA-CCC-ATG-GCC-AAC-CT-3′ (SEQ ID NO: 1) and 5′-CTA-GGG-TAG-AAT-CCG-AGT-ATG-TTG-3′ (SEQ ID NO: 7).

Nuclear DNA probes for use in the methods and reagents of the present invention may be prepared by a number of methods known to those of skill in the art. Suitable nuclear DNA probes may recognize any nuclear DNA target. In preferred embodiments of the methods and reagents of the invention, the nuclear DNA target is human papilloma virus (HPV) DNA.

Mitochondrial and nuclear DNA probes for use in the methods and reagents of the invention may be labeled using a number of methods and labels known to those of skill in the art. Suitable labels include, for example, enzymes, biotin, avidin, streptavidin, digoxygenin, luminescent agents, radiolabels, dyes, and haptens. Luminescent agents, depending upon the source of exciting energy, can be classified as radioluminescent, chemiluminescent, bioluminescent, and photoluminescent (including fluorescent and phosphorescent).

In one method of the invention, the label is a chemical reagent that yields an identifiable change when combined with the proper reactants. One example of a suitable chemical reagent is an enzyme, which when mixed with an appropriate enzyme substrate and cofactors, produces a detectable colored precipitate. A variety of different colored reaction products are commonly available using different enzyme substrates. Alkaline phosphatase is an example of an enzyme that has been used conventionally for the labeling of tissues. Other enzymes which may be used to practice the methods of the invention include, for example, horseradish peroxidase and galactosidase. Each of the enzymes that may be used to practice the methods of the invention has its own unique chromogenic system of specific substrates, co-factors, and resulting chromophoric reaction products.

In another method of the invention, mitochondrial and nuclear DNA probes are labeled with a fluorochrome moiety, which upon exposure to light of an appropriate wavelength, will become excited into a high-energy state and emit fluorescent light. Fluorochromes—substances that release significant amounts of fluorescent light—are generally divisible into two broad classes: intrinsic fluorescent substances and extrinsic fluorescent substances. Intrinsic fluorophores are comprised of naturally occurring biological molecules whose demonstrated ability to absorb exciting light and emit light of longer wavelengths is directly based on their internal structure and chemical formulation. Typical intrinsic fluorophores include, for example, proteins and polypeptides containing tryptophan, tyrosine, and phenylalamine. In addition, enzymatic cofactors such as NADH, FMN, FAD, and riboflavin are highly fluorescent. Extrinsic fluorophores, for the most part, do not occur in nature and have been developed for use as dyes to label proteins, immunoglobulins, lipids, and nucleic acids. This broad group includes, for example, fluorescein, rhodamine, and their isocyanate and isothiocyanate derivatives; dansyl chloride; naphthalamine sulfonic acids and their derivatives; acridine orange; proflavin; ethidium bromide; and quinacrine chloride. All of these are deemed suitable for use within the present invention.

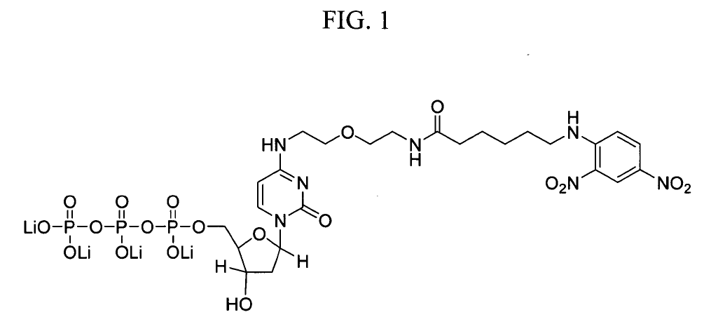

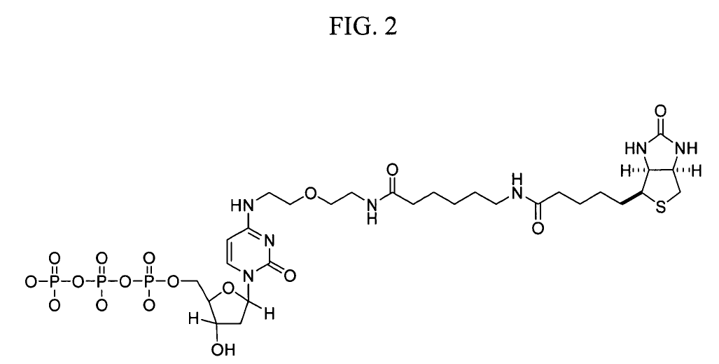

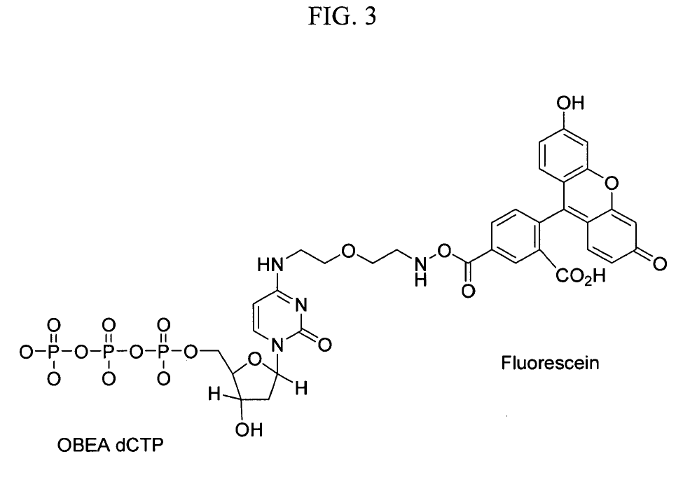

In preferred embodiments of the methods and reagents of the invention, the mitochondrial and nuclear DNA probes are labeled with fluoroscein, dinitrophenyl, biotin, or digoxygenin. These labels are incorporated into the mitochondrial and nuclear DNA probes during preparation of the probes using, for example, either a fluorescein-labeled nucleotide analog (fluorescein-dCTP) (FIG. 3), a dinitrophenyl (DNP)-labeled nucleotide analog (DNP-dCTP) (FIG. 1), or a biotinylated nucleotide analog (biotin-dCTP) (FIG. 2).

When monitoring chromogenic ISH analyses of nuclear DNA targets using a mitochondrial DNA probe as an internal control, the degree of hybridization of probes to the nuclear and mitochondrial DNA targets may be determined using identical haptens and detection systems, different haptens and identical detection systems, or different haptens and detection systems.

Because a cell's nucleus and mitochondria constitute distinct organelles occupying separate regions of the cytoplasm, the mitochondrial and nuclear DNA probes to be used in the methods and reagents of the invention may be labeled using the same detectable label. Alternatively, the mitochondrial and nuclear DNA probes may be labeled using different detectable labels.

The Examples, which follow, are illustrative of specific embodiments of the invention, and various uses thereof. They are set forth for explanatory purposes only, and are not to be taken as limiting the invention.

### EXAMPLE 1

### Preparation of Tissue and Cell Samples for Chromogenic In Situ Hybridization Analysis

Chromogenic in situ hybridization (CISH) analyses were performed using two human papilloma virus (HPV)-positive cell lines, CaSki (containing 200-600 copies of HPV 16) and HeLa (containing 10-50 copies of HPV 18), and one HPV-negative cell line (T24). Cell samples were fixed in 10% neutral buffered formalin, embedded in paraffin, and sectioned at 4-8 microns. Fixed cell samples were placed on Superfrost® Plus glass slides (VWR Scientific; West Chester, Pa.) prior to CISH analysis.

CISH analyses were also performed on cervical lesion cells of tissue biopsies and cervical smear samples prepared using commercially available liquid-based prep (LBP) systems from Cytyc Corp. (Boxborough, Mass.) and TriPath Imaging Inc. (Burlington, N.C.).

### EXAMPLE 2

### Preparation of Probes for Chromogenic In Situ Hybridization Analysis

HPV DNA probes for chromogenic in situ hybridization (CISH) analysis were prepared by cloning HPV DNA from genotypes 16, 18, 31, 33, 35, and 51 into plasmid vectors, as described in International Publication No. WO 00/24760.

Mitochondrial DNA probes for CISH analysis were prepared by PCR amplification using the Expand Long Template PCR System (Roche Molecular Biochemicals; Indianapolis, Ind.) and primers shown in Table I. Amplification reactions containing 500 μM of each dNTP, 5 units of Taq Polymerase, 0.3 μM of each primer, 50 mM KCl, 2.75 mM Mg₂Cl, 10 mM Tris-HCl, pH 8.5, and a DNA template from the human cell line, C33A, were performed at 94° C. for 2 minutes for one cycle and at 94° C. for 10 minutes, 55° C. for 30 minutes, and 68° C. for 15 minutes for 35 cycles. Amplification products were separated on a 0.6% agarose gel and analyzed using an α-imager. Products having the expected size were obtained using each of the five primer pairs (Table II). Each product was sequenced to confirm that the sequence was derived from human mitochondrial DNA.

TABLE I

| | | SEQ |
|---|---|---|
| | | ID |
| Primer | Sequence | NO: |
| L1 | 5′-AAC-ATA-CCC-ATG-GCC-AAC-CT-3′ | 1 |
| L2 | 5′-CCG-GGG-GTA-TAC-TAC-GGT-CA-3′ | 2 |
| L3 | 5′-CTC-TAG-AGC-CCA-CTG-TAA-AG-3′ | 3 |
| L4 | 5′-CAA-CAT-ACT-CGG-ATT-CTA-CCC-TAG-3′ | 4 |
| H1 | 5′-GGC-AGC-AGT-AAT-CAG-AGG-TG-3′ | 5 |
| H2 | 5′-GGG-GAA-GCG-AGG-TTG-ACC-TG-3′ | 6 |
| H3 | 5′-CTA--GGG-TAG-AAT-CCG-AGT-ATG-TTG-3′ | 7 |
| H4 | 5′-TGA-CCG-TAG-TAT-ACC-CCC-GG-3′ | 8 |

TABLE II

| Primer Pair | Primers | Product Size (bps) |
|---|---|---|
| 1 | L3; H4 | 16,434 |
| 2 | H2; L4 | 16,291 |
| 3 | L4; H4 | 10,830 |
| 4 | L3; H1 | 12,101 |
| 5 | L1; H3 | 10,624 |

HPV and mitochondrial DNA probes were column purified on QIAGEN columns (Quiagen Inc.; Valencia Calif.), and then labeled by nick translation using deoxycytosine triphosphate analogs (FIGS. 1-3; TriLink BioTechnologies, Inc.; San Diego, Calif.). DNase I was used to nick the probes, producing nicked fragments having an average size of 100-600 bp. Hapten-labeled dCTP was incorporated into the nicked fragments using the Kleno fragment of DNA Polymerase I. Unincorporated free nucleotides were then removed from the reaction mixture by ethanol precipitation or column purification on QIAGEN columns. Prior to CISH analysis, the purified and labeled probes were dissolved in formamide-based hybridization solution.

### EXAMPLE 3

### Analysis of Nuclear and Mitochondrial DNA Targets by Chromogenic In Situ Hybridization Using Identical Haptens and Detection Systems

CISH analysis of nuclear and mitochondrial DNA targets using identical haptens and detection systems was performed as follows. CaSki and cervical lesion cells of tissue biopsies were prepared as described in Example 1. Samples included formalin-fixed/paraffin-embedded tissues, formalin-fixed/paraffin-embedded tissue culture cell pellets, fixed tissue culture cells on Cytospin-prepared slides, and fixed cervical cells prepared with using the ThinPrep Pap Test specimen collection system (Cytyc Corp.). HPV and mitochondrial DNA probes were prepared and labeled with biotin-dCTP by nick translation as described in Example 2.

CISH was performed on a BenchMark® automated slide stainer (Ventana Medical Systems, Inc.). The degree of hybridization between the HPV and mitochondrial DNA probes and their respective targets was determined using one of two detection schemes. In the first detection scheme, the degree of hybridization between the HPV and mitochondrial DNA probes and their respective targets was determined using an HRP/DAB detection kit. This kit comprises horseradish peroxidase (HRP)-labeled streptavidin, which complexes with the biotin-labeled probes and reacts with the chromogen 3,3′-diaminobenzidine tetrahyrdochloride (DAB) to form a brown precipitate. In the second detection scheme, the degree of hybridization between the HPV and mitochondrial DNA probes and their respective targets was determined using alkaline phosphatase (AP)-streptavidin, which complexes with the biotin-labeled probes and dephosphorylates the substrate bromochloroindolyl (BCIP), which in turn reacts with the chromogen nitroblue tetrazolium (NBT) to form a blue precipitate or with the chromogen Azoic Diazo Component to form a red precipitate. CISH analysis of nuclear and mitchondrial DNA targets was performed on separate slides prepared from the same sample or on a single slide, with either simultaneous or sequential detection of hybridization between the HPV and mitochondrial DNA probes and their respective targets.

### EXAMPLE 4

### Analysis of Nuclear and Mitochondrial Targets by Chromogenic In Situ Hybridization Using Different Haptens and Identical Detection Systems

CISH analysis of nuclear and mitochondrial DNA targets using different haptens and identical detection systems was performed as follows. CaSki and cervical lesion cells of tissue biopsies were prepared as described in Example 1. Samples included formalin-fixed/paraffin-embedded tissues, formalin-fixed/paraffin-embedded tissue culture cell pellets, fixed tissue culture cells on Cytospin-prepared slides, and fixed cervical cells prepared with using the ThinPrep Pap Test specimen collection system (Cytyc Corp.). HPV probes were prepared and labeled with fluoroscein-dCTP or DNP-dCTP and mitochondrial DNA probes were prepared and labeled with biotin-dCTP by nick translation as described in Example 2.

CISH was performed on a BenchMark® automated slide stainer. The degree of hybridization between the HPV and mitochondrial DNA probes and their respective targets was determined using one of two detection schemes. In the first detection scheme, the degree of hybridization between the HPV and mitochondrial DNA probes and their respective targets was determined using an iVIEW™ Blue or V-Red detection kit from Ventana Medical Systems, Inc. Hybridization of the HPV and mitochondrial DNA probes was detected by first exposing hybridization complexes to a primary antibody capable of specifically binding the hapten-labeled probe, and then exposing the complexes to a biotinylated antibody capable of specifically binding the primary antibody. AP-streptavidin, which complexes with the biotinylated secondary antibody, was then added to the reaction mix. The AP-streptavidin dephosphorylates the substrate BCIP, which in turn reacts with the chromogen NBT to form a blue precipitate or with the chromogen Azoic Diazo Component to form a red precipitate. In the second detection scheme, the degree of hybridization between the HPV and mitochondrial DNA probes and their respective targets was determined using an HRP/DAB detection kit, as described above. With distinctive chromogen detection systems, one can perform CISH analysis of nuclear and mitchondrial DNA targets on a single slide, with either simultaneous or sequential detection of hybridization between the HPV and mitochondrial DNA probes and their respective targets.

### EXAMPLE 5

### Analysis of Nuclear and Mitochondrial DNA Targets by Chromogenic In Situ Hybridization Using Different Haptens and Detection Systems

CISH analysis of nuclear and mitochondrial DNA targets using different haptens and detection systems was performed as follows. CaSki and cervical lesion cells of tissue biopsies were prepared as described in Example 1. Samples included formalin-fixed/paraffin-embedded tissues, formalin-fixed/paraffin-embedded tissue culture cell pellets, and fixed tissue culture cells on Cytospin-prepared slides. HPV probes were prepared and labeled with fluoroscein-dCTP or DNP-dCTP and mitochondrial DNA probes were prepared and labeled with biotin-dCTP by nick translation as described in Example 2.

CISH was performed on a BenchMark® automated slide stainer. The degree of hybridization between HPV probes and nuclear DNA was determined using an AP/NBT/BCIP detection kit, as described in Example 4. The degree of hybridization between mitochondrial DNA probes and mitochondrial DNA was determined using an HRP/DAB detection kit as described in Example 3. CISH analysis of nuclear and mitchondrial DNA targets was performed on a single slide, with detection of mitochondrial DNA probe hybridization followed by detection of HPV probe hybridization.

### EXAMPLE 6

### Analysis of Nuclear DNA Target by Chromogenic In Situ Hybridization Using Mitochondrial DNA as an Internal Control

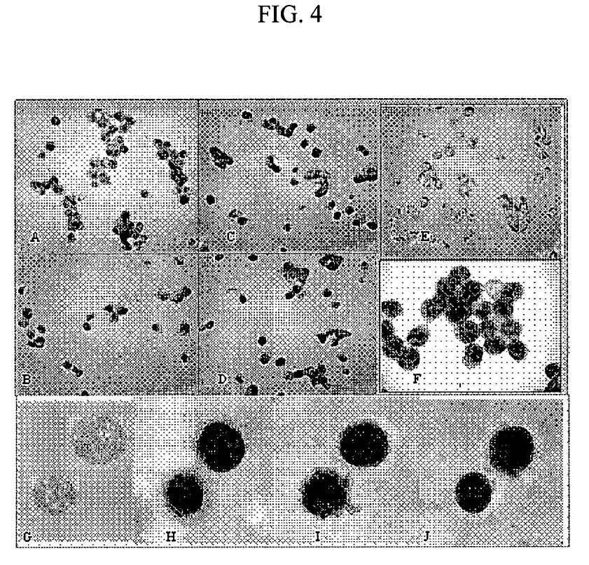

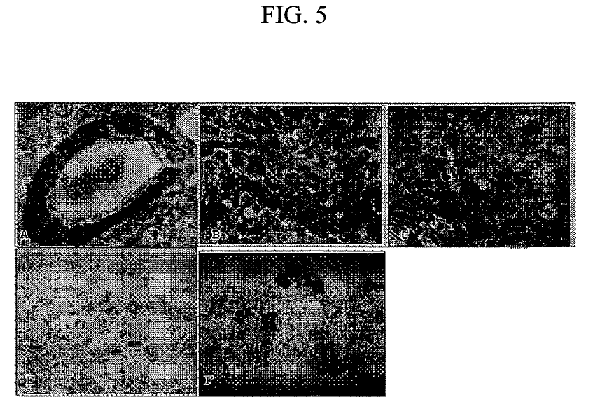

CISH analysis was performed using either HPV High Risk Tissue System Control Slides (Ventana Medical Systems, Inc.), which contain the CaSki, HeLa, and T24 cell lines, or clinical samples. Three different chromogenic detection systems were used to detect hybridization of HPV and mitochondrial DNA probes to cell and tissue samples. The results of CISH analysis using an AP/Azoic Diazo Component detection scheme are shown in FIGS. 4A-B, 4E-F, and 5D; the results of CISH analysis using an HRP/DAB detection scheme are shown in FIGS. 4C-D and 5A-B; and the results of CISH analysis using an AP/BCIP/NBT detection scheme are shown in FIGS. 5C and 5E.

In the cell samples, HPV staining was detected in both CaSki cells (FIG. 4H) and HeLa cells (FIG. 41) but not in T24 cells (FIG. 4J). In cell lines analyzed for both mitochondrial DNA and HPV staining, comparable mitochondrial DNA staining was observed in both CaSki cells (FIGS. 4A, 4C, and 4E) and T24 cells (FIGS. 4B and 4D), and HPV staining was observed only in CaSki cells (FIG. 4F).

While comparable mitochondrial DNA staining was observed in all clinical samples tested (FIGS. 5A, 5B, and 5D), HPV staining was observed only in a cervical lesion sample (FIG. 5E). Because comparable mitochondrial DNA staining was observed in both the cervical lesion sample (FIG. 5B) and cervical smear sample (FIG. 5D), the HPV staining results observed in these samples (FIGS. 5C and 5E) are reliable. Tissue or cell samples, therefore, that yield mitochondrial DNA staining but no HPV staining following CISH analysis can be considered as true HPV negatives, and tissue or cell samples that yield no mitochondrial DNA staining can be discarded as unreliable, regardless of whether HPV staining is positive or negative.

It should be understood that the foregoing disclosure emphasizes certain specific embodiments of the invention and that all modifications or alternatives equivalent thereto are within the spirit and scope of the invention as set forth in the appended claims.

1. A method for monitoring the quality of in situ hybridization analysis of a nuclear DNA target in a tissue or cell sample comprising:
(a) treating the tissue or cell sample to render chromosomal and extrachromosomal DNA present therein available for hybridization to complementary sequences;
(b) contacting the tissue or cell sample with a probe composition under hybridizing conditions, wherein the probe composition comprises:
(i) a nuclear DNA probe that is substantially complementary to the nuclear DNA target conjugated to a first detectable label; and
(ii) a mitochondrial DNA probe that is substantially complementary to a mitochondrial DNA target conjugated to a second detectable label;
(c) washing probe that does specifically hybridize to its target from the tissue or cell sample;
(d) assessing the degree of hybridization between:
(i) the nuclear DNA probe and the nuclear DNA target; and
(ii) the mitochondrial DNA probe and the mitochondrial DNA target;
wherein the degree of hybridization between the probes and their corresponding targets is assessed either simultaneously or sequentially;
(e) comparing the degree of hybridization between the mitochondrial DNA probe and the mitochondrial DNA target with the expected degree of hybridization between the mitochondrial DNA probe and the mitochondrial DNA target to determine the quality of in situ hybridization analysis of the nuclear DNA target.

2. The method of claim 1, wherein the nuclear DNA probe is substantially complementary to human papilloma virus DNA.

3. The method of claim 1, wherein the mitochondrial DNA probe is prepared by polymerase chain reaction using the amplimers:

| | | |
|---|---|---|
| (a) | 5′-CTC-TAG-AGC-CCA-CTG-TAA-AG-3′ | (SEQ ID NO: 3) |
| | and | |
| | 5′-TGA-CCG-TAG-TAT-ACC-CCC-GG-3′; | (SEQ ID NO: 8) |
| (b) | 5′-CAA-CAT-ACT-CGG-ATT-CTA-CCC-TAG-3′ | (SEQ ID NO: 4) |
| | and | |
| | 5′-GGG-GAA-GCG-AGG-TTG-ACC-TG-3′; | (SEQ ID NO: 6) |
| (c) | 5′-CAA-CAT-ACT-CGG-ATT-CTA-CCC-TAG-3′ | (SEQ ID NO: 4) |
| | and | |
| | 5′-TGA-CCG-TAG-TAT-ACC-CCC-GG-3′; | (SEQ ID NO: 8) |
| (d) | 5′-CTC-TAG-AGC-CCA-CTG-TAA-AG-3′ | (SEQ ID NO: 3) |
| | and | |
| | 5′-GGC-AGG-AGT-AAT-CAG-AGG-TG-3′; | (SEQ ID NO: 5) |
| | or | |
| (e) | 5′-AAC-ATA-CCC-ATG-GCC-AAC-CT-3′ | (SEQ ID NO: 1) |
| | and | |
| | 5′-CTA-GGG-TAG-AAT-CCG-AGT-ATG-TTG-3′. | (SEQ ID NO: 7) |

4. The method of claim 1, wherein the first detectable label and/or the second detectable label is biotin, avidin, streptavidin, digoxygenin, a luminescent agent, a radiolabel, a dye, an enzyme, or a hapten.

5. The method of claim 1, wherein the first detectable label and/or the second detectable label is fluoroscein, dinitrophenyl, biotin, or digoxygenin.

6. The method of claim 1, wherein the first detectable label and the second detectable label are the same.

7. The method of claim 1, wherein the first detectable label and the second detectable label are different.

8. A method for monitoring the quality of in situ hybridization analysis of a nuclear DNA target in a tissue or cell sample comprising:
(a) treating the tissue or cell sample to render chromosomal and extrachromosomal DNA present therein available for hybridization to complementary sequences;
(b) contacting the tissue or cell sample with either:
(i) a nuclear DNA probe that is substantially complementary to the nuclear DNA target conjugated to a first detectable label; or
(ii) a mitochondrial DNA probe that is substantially complementary to a mitochondrial DNA target conjugated to a first detectable label;
(c) washing probe that does specifically hybridize to its target in step (b) from the tissue or cell sample;
(d) assessing the degree of hybridization between the probe used in step (b) and its target;
(e) contacting the tissue or cell sample with either:
(i) a nuclear DNA probe that is substantially complementary to the nuclear DNA target conjugated to a second detectable label, provided that the tissue or cell sample was contacted with a mitochondrial DNA probe in step (b); or
(ii) a mitochondrial DNA probe that is substantially complementary to a mitochondrial DNA target conjugated to a second detectable label, provided that the tissue or cell sample was contacted with a nuclear DNA probe in step (b);
(f) washing probe that does specifically hybridize to its target in step (e) from the tissue or cell sample;
(g) assessing the degree of hybridization between the probe used in step (e) and its target; and
(h) comparing the degree of hybridization between the mitochondrial DNA probe and the mitochondrial DNA target with the expected degree of hybridization between the mitochondrial DNA probe and the mitochondrial DNA target to determine the quality of in situ hybridization analysis of the nuclear DNA target.

9. The method of claim 8, wherein the nuclear DNA probe is substantially complementary to human papilloma virus DNA.

10. The method of claim 8, wherein the mitochondrial DNA probe is prepared by polymerase chain reaction using the amplimers:

| | | |
|---|---|---|
| (a) | 5′-CTC-TAG-AGC-CCA-CTG-TAA-AG-3′ | (SEQ ID NO: 3) |
| | and | |
| | 5′-TGA-CCG-TAG-TAT-ACC-CCC-GG-3′; | (SEQ ID NO: 8) |
| (b) | 5′-CAA-CAT-ACT-CGG-ATT-CTA-CCC-TAG-3′ | (SEQ ID NO: 4) |
| | and | |
| | 5′-GGG-GAA-GCG-AGG-TTG-ACC-TG-3′; | (SEQ ID NO: 6) |
| (c) | 5′-CAA-CAT-ACT-CGG-ATT-CTA-CCC-TAG-3′ | (SEQ ID NO: 4) |
| | and | |
| | 5′-TGA-CCG-TAG-TAT-ACC-CCC-GG-3′; | (SEQ ID NO: 8) |
| (d) | 5′-CTC-TAG-AGC-CCA-CTG-TAA-AG-3′ | (SEQ ID NO: 3) |
| | and | |
| | 5′-GGC-AGG-AGT-AAT-CAG-AGG-TG-3′; | (SEQ ID NO: 5) |
| | or | |
| (e) | 5′-AAC-ATA-CCC-ATG-GCC-AAC-CT-3′ | (SEQ ID NO: 1) |
| | and | |
| | 5′-CTA-GGG-TAG-AAT-CCG-AGT-ATG-TTG-3′. | (SEQ ID NO: 7) |

11. The method of claim 8, wherein the first detectable label and/or the second detectable label is biotin, avidin, streptavidin, digoxygenin, a luminescent agent, a radiolabel, a dye, an enzyme, or a hapten.

12. The method of claim 8, wherein the first detectable label and/or the second detectable label is fluoroscein, dinitrophenyl, biotin, or digoxygenin.

13. The method of claim 8, wherein the first detectable label and the second detectable label are the same.

14. The method of claim 8, wherein the first detectable label and the second detectable label are different.

15. A reagent for in situ hybridization detection of a nuclear DNA target and a mitochondrial DNA target in a tissue or cell sample comprising:
(a) a nuclear DNA probe that is substantially complementary to the nuclear DNA target conjugated to a first detectable label; and
(b) a mitochondrial DNA probe that is substantially complementary to the mitochondrial DNA target conjugated to a second detectable label.

16. The reagent of claim 15, wherein the nuclear DNA probe is substantially complementary to human papilloma virus DNA.

17. The reagent of claim 15, wherein the mitochondrial DNA probe is prepared by polymerase chain reaction using the amplimers:

| | | |
|---|---|---|
| (a) | 5′-CTC-TAG-AGC-CCA-CTG-TAA-AG-3′ | (SEQ ID NO: 3) |
| | and | |
| | 5′-TGA-CCG-TAG-TAT-ACC-CCC-GG-3′; | (SEQ ID NO: 8) |
| (b) | 5′-CAA-CAT-ACT-CGG-ATT-CTA-CCC-TAG-3′ | (SEQ ID NO: 4) |
| | and | |
| | 5′-GGG-GAA-GCG-AGG-TTG-ACC-TG-3′; | (SEQ ID NO: 6) |
| (c) | 5′-CAA-CAT-ACT-CGG-ATT-CTA-CCC-TAG-3′ | (SEQ ID NO: 4) |
| | and | |
| | 5′-TGA-CCG-TAG-TAT-ACC-CCC-GG-3′; | (SEQ ID NO: 8) |
| (d) | 5′-CTC-TAG-AGC-CCA-CTG-TAA-AG-3′ | (SEQ ID NO: 3) |
| | and | |
| | 5′-GGC-AGG-AGT-AAT-CAG-AGG-TG-3′; | (SEQ ID NO: 5) |
| | or | |
| (e) | 5′-AAC-ATA-CCC-ATG-GCC-AAC-CT-3′ | (SEQ ID NO: 1) |
| | and | |
| | 5′-CTA-GGG-TAG-AAT-CCG-AGT-ATG-TTG-3′. | (SEQ ID NO: 7) |

18. The reagent of claim 15, wherein the first detectable label and/or the second detectable label is biotin, avidin, streptavidin, digoxygenin, a luminescent agent, a radiolabel, a dye, an enzyme, or a hapten.

19. The reagent of claim 15, wherein the first detectable label and/or the second detectable label is fluoroscein, dinitrophenyl, biotin, or digoxygenin.

20. The reagent of claim 15, wherein the first detectable label and the second detectable label are the same.

21. The reagent of claim 15, wherein the first detectable label and the second detectable label are different.

22. The reagent of claim 15, wherein the reagent comprises a kit in which the nuclear DNA probe is provided in a first container and the mitochondrial DNA probe is provided in a second container.


SEQUENCE LISTING (8 sequences)

SEQ ID NO: 1 - DNA, 20 bp, Artificial
  PCR amplimer
aacatacccatggccaacct

SEQ ID NO: 2 - DNA, 20 bp, Artificial
  PCR amplimer
ccgggggtatactacggtca

SEQ ID NO: 3 - DNA, 20 bp, Artificial
  PCR amplimer
ctctagagcccactgtaaag

SEQ ID NO: 4 - DNA, 24 bp, Artificial
  PCR amplimer
caacatactcggattctaccctag

SEQ ID NO: 5 - DNA, 20 bp, Artificial
  PCR amplimer
ggcaggagtaatcagaggtg

SEQ ID NO: 6 - DNA, 20 bp, Artificial
  PCR amplimer
ggggaagcgaggttgacctg

SEQ ID NO: 7 - DNA, 24 bp, Artificial
  PCR amplimer
ctagggtagaatccgagtatgttg

SEQ ID NO: 8 - DNA, 20 bp, Artificial
  PCR amplimer
tgaccgtagtatacccccgg